(19) United States
(12) Patent Application Publication
(10) Pub. No.: US 2003/0139731 A1
(43) Pub. Date: Jul. 24, 2003

(54) Device for transcutaneous drug delivery and uses therefor

(76) Inventors: Kevin Marchitto, Golden, CO (US); Stophen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/349,696

(22) Filed: Jan. 23, 2003

Related U.S. Application Data
(60) Provisional application No. 60/351,329, filed on Jan. 23, 2002.
(60) Provisional application No. 60/351,253, filed on Jan. 23, 2002.

(51) Int. Cl. A61K 9/22

(52) U.S. Cl. 604/890.1

(57) ABSTRACT

Provided herein is a device for increasing the rate of permeation of a biological substance through biomembranes of an individual comprising an active electrode having a proximal end and a distal end such that the active electrode delivers a high frequency voltage to the biomembrane and a return electrode which is located distally to the active electrode. Also provided are methods of using the device.

### CROSS-REFERENCE TO RELATED APPLICATIONS

This non-provisional application claims priority to provisionals U.S. Ser. No. 60/351,329, filed Jan. 23, 2002, now abandoned and U.S. Ser. No. 60/351,251, filed Jan. 23, 2002, now abandoned.

### BACKGROUND OF THE INVENTION

1. Field of the Invention

The present invention relates generally to the fields of biomedical physics and drug delivery. More specifically, the present invention provides a device and methods for improving the permeation rates of substances across biological membranes.

2. Description of the Related Art

Various methods have been used for facilitating the delivery of compounds across the skin and other membranes. Iontophoresis uses an electric current to increase the permeation rate of charged molecules. However, iontophoresis is dependent on charge density of the molecule and has further been known to cause burning in patients. Use of ultrasound has also been tested whereby application of ultrasonic energy to the skin results in a transient alteration of the skin, which leads to an increased permeability to substances. Electromagnetic energy produced by lasers may be used to ablate the stratum corneum in order to make the skin more permeable to pharmaceutical substances (U.S. Pat. No. 4,775,361 ). Impulse transients generated by lasers or by mechanical means may be used to make alterations in epithelial layers that result in improved permeation of compounds (U.S. Pat. No. 5,614,502 ).

In general, permeation of drugs through the skin occurs at a very slow rate, if at all. The primary rate limiting step in this process is the passage of these compounds through the outermost layer of skin, called the stratum corneum. The stratum corneum is a very thin layer of dead cells that acts as an impermeable layer to matter on either side of this layer. The stratum corneum primarily provides the skin's barrier function. It has long been recognized that loss or alteration of the stratum corneum results in increased permeability to many substances; materials can more easily diffuse into or out of the skin. Alternatively, compounds referred to as permeation enhancers, e.g., alcohol or drug carriers such as liposomes, can be used, with some success, to penetrate the stratum corneum. The barrier function of the skin presents a very significant problem to pharmaceutical manufacturers interested in topical administration of drugs, or in cutaneous collection of bodily fluids.

Electrosurgery is a method whereby tissue coagulation and/or dissection can be effected. In electrosurgery radiofrequency (RF) current is applied to tissue by an active electrode. In a bipolar system, the current is passed through tissue between two electrodes on the same surgical instrument, such as a forceps. In a monopolar system, a return-path (ground) electrode is affixed in intimate electrical contact with some part of the patient. Because of the importance of the ground electrode providing the lowest impedance conductive path for the electrical current, protection circuits monitoring the contact of the ground with the patient are often employed wherein an increase in ground electrode-skin impedance results in the instrument shutting down. A desired alteration in the tissue, usually coagulation or cutting, can be made by manipulating the treatment electrode shape, the electrode position (contact or non-contact) with respect to the tissue surface, frequency and modulation of the RF current, power of the RF current and the length of time for which it is applied to the tissue surface, and peak-to-peak voltage of the RF current with respect to the tissue type.

For example, decreasing electrode size translates into increased current density in the tissue proximal to the electrode and so a more invasive tissue effect, such as dissection as compared to coagulation, is realized. Similarly, if the electrode is held close to the tissue but not in contact, then the area of RF-tissue interaction is smaller as compared to the area when the electrode is in contact with the tissue, therefore, the effect on the tissue is more invasive. By changing the waveform of the applied RF current from a continuous sinusoid to packets of higher peak voltage sinusoids separated by dead time (i.e. a duty cycle of 6%), then the tissue effect can be changed from dissection to coagulation. Increasing the voltage of the waveform increases the invasiveness of the tissue effect, and the longer the tissue is exposed to the RF, the greater the tissue effect. Finally, different tissues respond to RF differently because of their different electrical conductive properties, concentration of current carrying ions, and different thermal properties. In a typical electrosurgical system, RF frequencies of 300 kHz to 4 MHz are used since nerve and muscle stimulation cease at frequencies beyond 100 kHz.

Devices incorporating radio frequency electrodes for use in electrosurgical and electrocautery techniques are described in Rand¹ et al. and U.S. Pat. Nos. 5,281,216; 4,943,290; 4,936,301; 4,593,691; 4,228,800; and 4,202,337.

U.S. Pat. Nos. 4,943,290 and 4,036,301 describe methods for injecting non-conducting liquid over the tip of a monopolar electrosurgical electrode to electrically isolate the electrode, while energized, from a surrounding electrically conducting irrigant.

U.S. Pat. Nos. 5,195,959 and 4,674,499 describe monopolar and bipolar electrosurgical devices, respectively, that include a conduit for irrigating the surgical site.

U.S. Pat. Nos. 5,217,455, 5,423,803, 5,102,410, 5,282,797, 5,290,273, 5,304,170, 5,312,395, 5,336,217 describe laser treatment methods for removing abnormal skin cells, such as pigmentations, lesions, soft tissue and the like.

U.S. Pat. Nos. 5,445,634 and 5,370,642 describe methods for using laser energy to divide, incise or resect tissue during cosmetic surgery. U.S. Pat. No. 5,261,410 is directed to a method and apparatus for detecting and removing malignant tumor tissue.

U.S. Pat. Nos. 5,380,316, 4,658,817, 5,389,096, International Publication WO 94/14383 and European Patent Application No. 0 515 867 describe methods and apparatus for percutaneous myocardial revascularization. These methods and apparatus involve directing laser energy against the heart tissue to form transverse channels through the myocardium to increase blood flow from the ventricular cavity to the myocardium.

Devices and methods in U.S. Pat. Nos. 5,683,366, 5,697,536, 6,228,078, and 5,888,198 describe bipolar and monopolar RF electrosurgical devices that use a method of tissue disintegration as a means to ablate tissue prior to myocardial revascularization, tissue resurfacing or other surgical procedures.

Devices and methods for drug delivery using laser ablation systems have been described. U.S. Pat. No. 6,251,100 provides an improved method of administering a pharmaceutical composition, such as an anesthetic through the skin of a patient without the use of a sharp or needle. This method includes the step of irradiating the stratum corneum of a region of the skin of the patient using a laser. By a selection of parameters, the laser irradiates the surface of the skin precisely to a selectable depth, without causing clinically relevant damage to healthy proximal tissue. A pharmaceutical composition is then applied to the region of irradiation. International Publication WO 00/57951 describes the use of non-ionizing energy, including lasers, to improve methods of administering pharmaceuticals in tissues, including the skin.

The inventors have recognized a need in the art for a device and improved methods of controllably facilitating permeation of substances across tissue membranes while minimizing the impact of the method on the tissue membrane. The prior is deficient in the lack of a device and methods to use radiofrequency current to controllably alter the permeability of a biological membrane to a pharmaceutical or other biological molecule. The present invention fulfills this longstanding need and desire in the art.

### SUMMARY OF THE INVENTION

One embodiment of the present invention provides a device for increasing the rate of permeation of a biological substance through a biomembrane of an individual comprising an active electrode which has a proximal end and a distal end where the active electrode delivers a high frequency voltage to the biomembrane and a return electrode located distally to said active electrode.

Another embodiment of the present invention provides a method of increasing the rate of permeation of a substance through the skin of an individual comprising the steps of applying a high frequency voltage with the device described herein to a target area on the skin of the individual where the target area on the skin comprises a substance applied externally on or located internally to the target area and successively reducing the stratum corneum of the skin with the application of the high frequency voltage thereby increasing the rate of permeation of the substance through the skin of the individual.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 depicts a block diagram of the treatment device.

FIG. 2 depicts a diagram of a coaxial electrode design.

FIG. 3 depicts a diagram of a needle electrode design.

FIG. 4 depicts a diagram of electrodes made on a copper-clad printed circuit board.

FIGS. 5A-5C depict diagrams of electrodes made with conductive ink or conductive tape.

FIG. 6 is a photomicrograph of human skin treated in vitro with the device of the present invention.

FIG. 7 is a graph of the enhancement of the permeation of lidocaine through human skin in vitro using the device of the present invention.

FIG. 8 is a graph of the enhancement of the permeation of fentanyl through human skin in vitro at various times post-drug-application treated with the device of the present invention.

FIG. 9 is a graph of the in vitro enhancement of the permeation of a variety of drugs through human skin treated with the device of the present invention.

### DETAILED DESCRIPTION OF THE INVENTION

One embodiment of the present invention provides a device for increasing the rate of permeation of a biological substance through a biomembrane of an individual comprising an active electrode which has a proximal end and a distal end where the active electrode delivers a high frequency voltage to the biomembrane and a return electrode located distally to said active electrode.

In an aspect of this embodiment the device may further comprise a control means that independently controls current flow from the active electrode. Impedance between the active electrode at a target site on the biomembrane and the return electrode determines the current flow. The control means may generate a high intensity electric field at the distal end of the active electrode. A representative action of this electric field is to cause a molecular disruption of necrotic or dead tissue, biomolecules or cells at the target site on the skin.

In this embodiment the active electrode and the return electrode may comprise a coaxial electrode, needles, a printed-circuit, conductive-ink or conductive tape. Furthermore, the conductive ink or the conductive tape may be positioned on an electrically insulative material that is placed on the skin. The insulative material insulates that part of the skin from those parts of the conductive ink or the conductive tape which is not in immediate contact with a target site on the skin.

In one aspect the active electrode may be moved over the target site on the biomembrane during delivery of the voltage. A representative area of the target site is about 0.1 cm² to about 20 cm². Optionally, the active electrode may comprise an electrode array having a plurality of isolated electrode terminals. In another aspect the active and return electrodes are in a patch. The active electrode in the patch may comprise a transducer.

In other aspects the device may have a safety interlock which regulates the operation of the device. A container may also b e located at the distal end of the active electrode and may optionally be integrated with the safety interlock. Additionally, the device may contain a means to apply an electrically conductive or insulating fluid. The conductive or insulating fluid may contain the substance to be applied to the target site.

The substances used in this embodiment may be biological molecules such as pharmaceutical compounds. Representative examples of such compounds are nitroglycerin, an anti-nauseant, an analgesic, a hormone, a steroidal antiinflammatory agent, a non-steroid antiinflammatory agent, a chemotherapeutic agent, an anti-cancer agent, an immunogen, an anti-viral agent or an anti-fungal agent. The anti-nauseant may be scopolamine. Examples of an antiobiotic are tetracycline, streptomycin, sulfa drugs, kanamycin, neomycin, penicillin, or chloramphenicol. Examples of a hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin.

Additionally, the substances of the present invention may be interstitial fluid or a diagnostic reagent. These substances may be removed from tissue using the methods disclosed herein. A representative example of a use for interstitial fluid is to measure analytes.

In another embodiment of this invention there is provided a method of increasing the rate of permeation of a substance through the skin of an individual comprising the steps of applying a high frequency voltage with the device described supra to a target area on the skin of the individual where the target area on the skin comprises a substance applied externally on or located internally to the target area and successively reducing the stratum corneum of the skin with the application of the high frequency voltage thereby increasing the rate of permeation of the substance through the skin of the individual. All aspects of the device and substances used in this embodiment are as described supra. Additionally, the substance may be interstitial fluid. A representative use of the interstitial fluid is to measure analytes. The substance may also comprise a diagnostic material.

The present invention provides a device and methods for improving the permeability of the skin or other biomembranes to certain substances. Targets associated with tissue interfaces are made permeable to diagnostic and therapeutic substances. The device and methods disclosed herein can improve the permeation rate of pharmaceuticals across a biological membrane into an individual or can increase the diffusion of substances out of a tissue of the individual. The system allows the operator to cause molecular alterations in necrotic tissue or dead cells present in, for example, the stratum corneum by selectively applying electromagnetic energy, e.g., radiofrequency energy, to the skin in the presence of a desired substance prior to its application or prior to withdrawal of compounds from the tissues. The transient or sustained molecular alteration of membranes and tissue interfaces induced by high frequency electromagnetic energy or by the physical products of the interaction of the electromagnetic energy and matter improve permeability to the particular substance. The system is useful for delivery of drugs, diagnostic agents and for extraction of blood chemicals and gases for diagnostics.

The devices described herein can be used to reduce the stratum corneum in order to create a site which is substantially more permeable to substances, including drugs and other medically useful compounds. As successive layers of the stratum corneum are removed, permeation generally increases until a maximum rate of permeation or flux occurs at which point the stratum corneum is completely removed. Thus, by manipulating the depth or degree of reduction, one may control the flux of a certain substance.

Once the barrier is reduced, a drug may be supplied to the surface of the target. Alternatively, the drug may be supplied in the electrically conductive or insulating liquid during the ablation process or the drug may be supplied from a reservoir independent of the electrically conductive or insulating liquid and applied after the process of ablation occurs. An advantage to this device and this method is that the ablation process occurs at a relatively low temperature, thus minimizing damage to surrounding tissue or to the drug itself. The insulating liquid also reduces the conduction of current into the tissue.

Additionally, an advantage of the present method of transcutaneous drug delivery, particularly over previous methods involving lasers, is that the high frequency voltage can be continuously or intermittently applied to the target site to reduce the stratum corneum. Thus, the site can be treated over long periods of time, thereby slowing or stopping the healing process that would otherwise replace the stratum corneum. Intermittent pulses can b e delivered as the layers are replaced, thereby maintaining the increased permeability at the site.

The present device and methods can be used for transport of a variety of systemically or locally acting pharmaceutical substances. For example, these substances may be nitroglycerin and anti-nauseants such as scopolamine, antibiotics such as tetracycline, streptomycin, sulfa drugs, kanamycin, neomycin, penicillin, or chloramphenicol. Various hormones such as parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin, steroidal or non-steroidal anti-inflammatory agents, and systemic antibiotic, antiviral or antifungal agents may also b e transported.

The device may be in a patch or in a probe form. An active electrode is placed in proximity to the target tissue site and a return electrode is positioned distal from the first electrode so a current flow path is generated between the two electrodes when a high frequency power source is applied. The high frequency power source may be distal or integral to the unit. Either one or both electrodes may be placed within an electrically conducting liquid, such as isotonic saline, or an electrically insulating fluid such as deionized water. Additionally, either one or both electrodes may have an insulative material positioned between the skin and that part of the electrode(s) not in contact with an electrically conducting liquid.

High frequency voltage is applied between the active and the return electrode through the current flow path created by the electrically conducting or insulating liquid in either a bipolar or monopolar manner. Preferably, the current flow path may be created in the system, between the patch or probe and the skin whereby the target site and return electrode are bathed in an electrically conductive or insulating solution. Alternatively, the probe may be scanned across an area of the skin to expand the area useful for treatment or across the patch designed to encompass a large surface area. In both cases, the return electrode is spaced from the active electrode and shielded by an insulating material, thus reducing the risk of exposure of the return electrode to nearby tissue.

The high frequency voltage is believed to result in the formation of an electric field at the fluid supplied to the target site, which in turn generates a high energy plasma of electrons and, possibly, photons, which vaporize or alter the adjacent dead or necrotic cells. Precise control over the process results from manipulation of the high frequency voltage (voltage, frequency, duty cycle, pulse-width, pulse shape) with respect to changes in the impedence across the target site. The device may be optionally controlled with a feedback device that monitors the impedence of the target, allowing for automated control based on the variance in the impedence. The device may be further controlled through the continual or intermittent supply of the electrically conductive fluid. This continued or intermittent treatment ensures that the site of treatment is maintained at the more permeable state.

A safety interlock may be affixed to the distal end of the active electrode, or integrated into the patch such that the device cannot be utilized unless the interlock is engaged, and only under proper use. For example, the interlock could be mechanical, electrical or optical. In the “on” position (engaged or disengaged), the device may be operational. In the “off” position, the device would fail to be operational.

A container may be attached to the distal end of the active electrode such as to contain the spark and collect ablated tissue. The container may be permanent or disposable. Alternatively, in a patch device, the container would be equivalent to a disposable or non-disposable component that is in contact with the skin. The container may be modified to hold, or receive through an opening, a pharmaceutical or other substance, which may then be delivered simultaneously, or shortly after irradiation occurs. The container may be integral to, or function independently of a safety interlock.

The device may be used to control delivery of pharmaceuticals. In general, the impedence of the skin can approach values as high as 10⁸ ohms. As successive layers of the stratum corneum are removed, this impedance can drop to a fraction of that value. This drop in impedence can be monitored as a measure of the degree of the process. Another aspect of the invention is that, with the other parameters set, the depth of treatment can be precisely controlled by continuously monitoring the impedence across the target area, and causing a feedback loop whereby the process is halted when a desired endpoint is met. Therefore, various settings on the device can be adjusted to allow successive reduction of the stratum corneum.

This method of delivering a pharmaceutical creates a variable size zone in which the target is irradiated, and minimizes the risk of thermal necrosis on tissues surrounding the target site. A practical round irradiation site can range from 0.1-5.0 cm in diameter. After irradiation, the drug can then be applied directly to the skin or in a pharmaceutically acceptable formulation such as a cream, ointment, lotion or patch. One of ordinary skill in the art would have no trouble in determining how to formulate the drug for this topical application.

Alternatively, the delivery zone can be enlarged by strategic location of the irradiation sites and by the use of multiple sites. For example, in the case of an anesthetic, a region of the skin may be anesthetized by first scanning the desired area with the active electrode such that the treatment occurs over a larger surface area. Or, a patch device can incorporate a single large transducer, or multiple transducer (electrodes) such that the surface area of treatment can be quite large. The electrodes in the multiple transducer format can be excited in a multiplex fashion in order to save energy. An important advantage of the device and method is that the size of the treatment site can be conveniently modulated. Further, the size and shape of the treatment site may be altered through the use of multiple probes, or through the size and shape of the probes.

The device also may be used to control toxicity of pharmaceuticals delivered thereby. One of the limitations of transcutaneous delivery of drug formulations is that the drug can be toxic at high doses, and therefore must be modulated to permeate the skin at a controlled rate. In the present case, modulation may occur by limiting the depth of the treatment. Depth of treatment can be correlated with the change in impedence across the site as the stratum corneum is reduced. When a desired depth is reached, the device can be shut down. Also, the skin impedance can be used to modulate the electromagnetic energy in such a way that the process becomes curtailed as the impedance of the skin drops.

The present invention provides a means for treating local pain or infections, or for application of a substance to a small specified area, directly, thus eliminating the need to provide high, potentially toxic amounts systemically through oral or intravenous administration. Locally acting pharmaceuticals such as alprostadil (for example, Caverject from Pharmacia & Upjohn), various antibiotics, antiviral or antifungal agents, or chemotherapy or anti-cancer agents, can be delivered using this method to treat regions proximal to the delivery site. Protein or DNA based biopharmaceutical agents can also be delivered using this method.

The device also may be used to deliver immunogens. Antigens derived from a virus, bacteria or other agent which stimulates an immune response can be administered through the skin for immunization purposes. The antigen is delivered through the outer layers of the stratum corneum, either singly or multiply, and the immunogen is provided in an appropriate formulation. For booster immunizations, where delivery over a period of time increases the immune response, the immunogen can be provided in a formulation that penetrates slowly through the treatment site, but at a rate faster than possible through unaltered skin.

Additionally, anti-inflammatory drugs may be delivered. Analgesics and other non-steroidal anti-inflammatory agents, as well as steroidal anti-inflammatory agents, may be caused to permeate through reduced stratum corneum to locally affect tissue within proximity of the irradiated site. For example, anti-inflammatory agents such as Indocin (Merck & Co.), a non-steroidal drug, are effective agents for treatment of rheumatoid arthritis when taken orally, yet sometimes debilitating gastrointestinal effects can occur. By administering such agents through laser-assisted perforation or alteration sites, these potentially dangerous gastrointestinal complications may be avoided. Further, high local concentrations of the agents may be achieved more readily near the site of irradiation as opposed to the systemic concentrations achieved when orally administered.

It is contemplated that fluids, gases or other biomolecules may be drawn from the individual. The devices provided herein can be used to alter the stratum corneum to improve the collection of fluids, gases or other biomolecules through the skin. The fluid, gas or other biomolecule can be used for a wide variety of tests. For example, the technique of the present invention may be used to improve the ability to sample extracellular fluid in order to quantify glucose or other analytes. Glucose is present in the extracellular fluid in the same concentration as (or in a known proportion to) the glucose level in blood².

The alteration of the stratum corneum causes a local increase in the water loss through the skin (referred to as transepidermal water loss, or TEWL). With successive reduction of the stratum corneum, there is a corresponding increase in water loss. The tape strip data is a positive control that proves that the measurement is indeed sensitive to increased skin water evaporation.

The device can alter the tissue without ablation thereof. The technique of successive removal of layers of dead or necrotic cells of the stratum corneum provides several advantages. Preferably, the stratum corneum is reduced, but not removed, so that its structural and biochemical makeup still permit drugs to permeate. Therefore, the skin after irradiation still presents a barrier, albeit reduced, to external factors such as viruses and chemical toxins. Less energy is required for reduction than is required to entirely remove the stratum corneum, thus smaller and cheaper devices can be used. The technique also minimizes the damage to surrounding tissues providing a more rapid and efficient replacement of the stratum corneum.

As described herein, the invention provides a number of therapeutic advantages and uses. Embodiments of the present invention are better described below with reference to the Figures, however, such description or reference is not meant to limit the present invention in any fashion. The embodiments and variations described in detail herein are to be interpreted by the appended claims and equivalents thereof.

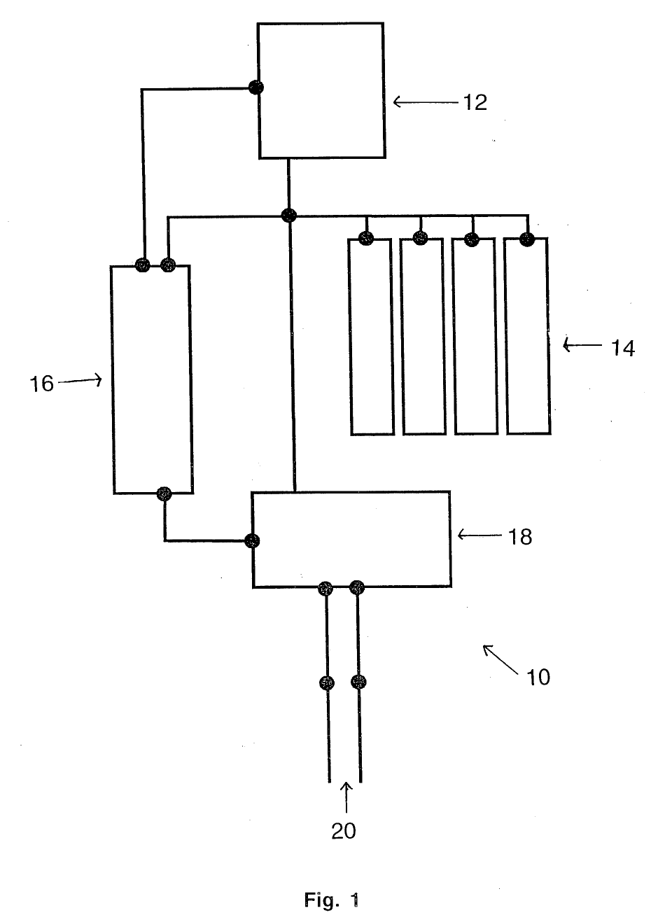

The treatment device described herein produces a controlled, adjustable high-voltage pulse that is applied to an electrode that is in contact with, or positioned close to, a biological membrane surface, such as the skin. As depicted in FIG. 1 the device 10 comprises an electrical energy source 14, e.g., four AA batteries, which powers a microprocessor and control electronics 12 and a pulse-forming network 16. The output of the pulse-forming network 16 is connected to a transformer 18 that increases the voltage amplitude of the pulse. The voltage produced in each pulse exceeds the dielectric breakdown voltage of air, i.e., approximately 3 kV/mm. An electrode pair 20 is attached to the output of the transformer 18. The resulting output is controllable as a burst of 1 to about 16 electrical pulses of about 400 ns to about 150 μs duration at a repetition rate of about 25 Hz to about 10 kHz. The discharge energy is related to the pulse length and varies around 10 mJ at about 150 μs.

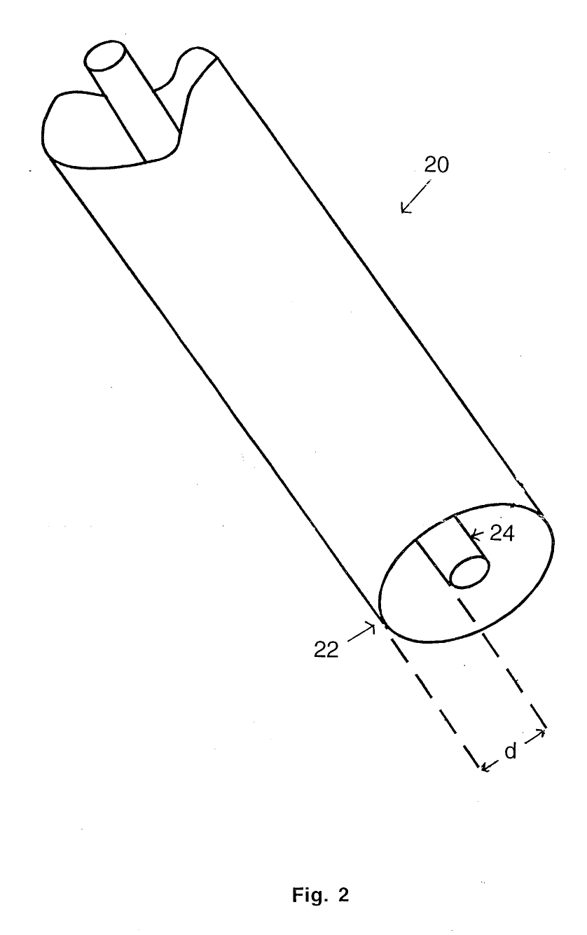

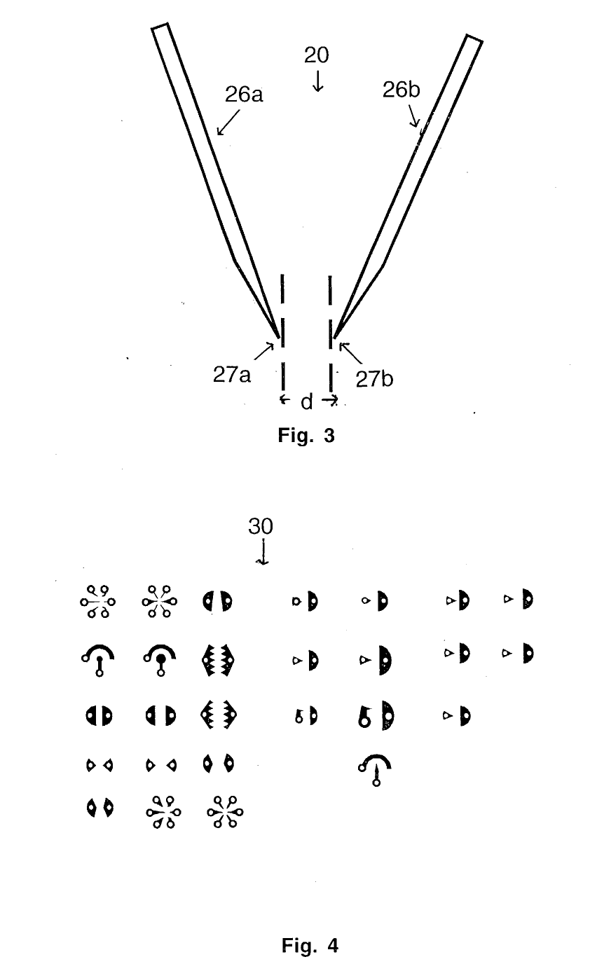

Regarding the electrode pair 20 depicted in FIG. 1 and without being limiting various designs may be used in constructing the electrode pair 20. As shown in FIG. 2, the electrode pair 20 comprises a solid wire 24 positioned coaxially within the lumen of a metal tube 22. The metal wire and tube can be made up of highly conductive materials such as copper or aluminum, or can be constructed of a less conductive metal such as stainless steel. The radial distance d between the wire 24 and the metal tube 22 is approximately 2 mm. As depicted in FIG. 3, the electrode pair 20 may comprise two 20 gauge stainless-steel hypodermic needles 26a,b. The needles 26a,b each have a sharp end 27a,b which are proximately positioned on the skin surface (not shown) a distance d of no more than about 2 mm.

It is contemplated that alternative electrode designs may be used in the device of the present invention. Electrode pairs may be manufactured of copper clad to printed circuit board substrates. FIG. 4 depicts a mask 30 used to create the electrodes using photolithographic techniques.

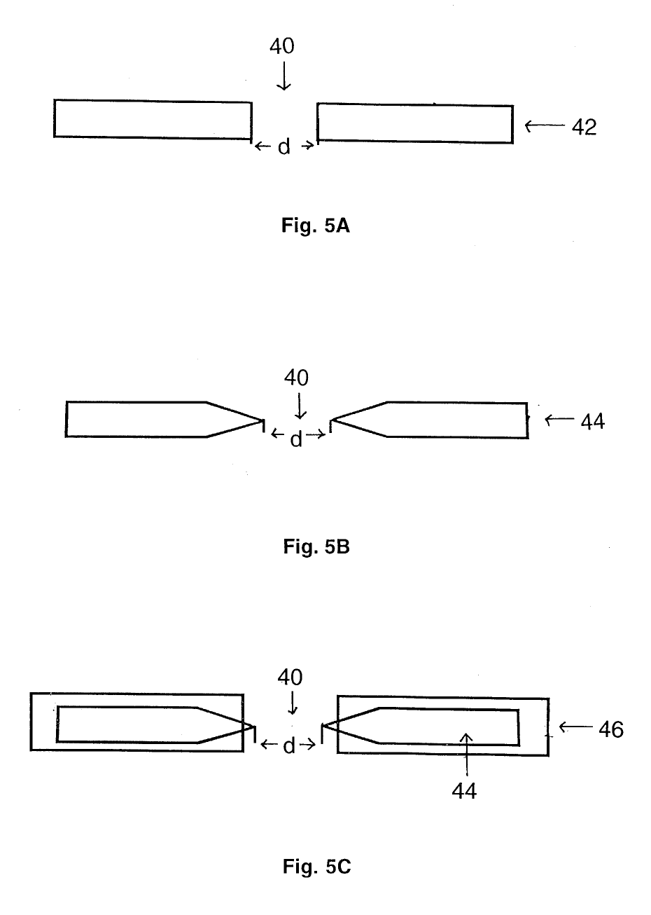

Alternative electrode designs are depicted in FIGS. 5A-5C. The electrode pair 40 may comprise copper conductive tape (Ted Pella, Inc, Redding Calif.), disposable printed electrode material or conductive silver ink (Conductive Compounds, Inc., Londonderry, N.H.) which are positioned directly against the skin (not shown) along the longitudinal axes of the electrode pair 40. As shown in FIGS. 5A-5B the electrode pair may have shapes 42 and 44, respectively. As in FIG. 5C, an electrically insulating material 46, e.g., adhesive tape, may be positioned between any of the conductive electrodes 40 having either of the shapes 42, 44 described and the skin such that an end of the electrodes 40 remains exposed. Thus the majority of the electrodes 40 is insulated from the skin surface. Again, in FIGS. 5A-5C, all the electrode pairs 40 are positioned on the skin surface a distance d not to exceed about 2 mm.

The following examples are given for the purpose of illustrating various embodiments of the invention described supra and are not meant to limit the present invention in any fashion.

### EXAMPLE 1

Durability of Electrode Material Subjected to Applied Plasma

The output of the treatment device was electrically connected to the disposable printed electrode material and was set to produce a single pulse of electrical energy at the highest energy available, which is minimally 10 mJ. The geometry of the printed electrode material was two rectangles, approximately 10×2 mm and separated by about 2 mm. The plasma produced was observed through a dissection microscope. Each pulse eroded the printed electrode material slightly. The cathode or anode electrode eroded at different rates depending on their polarity and geometric shape. The plasma in subsequent pulses propagated along the electrode material with each pulse until the entire electrode was critically degraded, whereupon no more plasma could be produced unless the electrode was replaced. Similar results were obtained when conductive ink was used, except that that rate of erosion could be reduced with increasing electrode thickness.

### EXAMPLE 2

Ablative Characteristics of Various Electrodes

The output of the treatment device was electrically connected to the coaxial electrode and was set to produce a single pulse of electrical energy at the higher energy available. The electrode was gently pressed against the skin on the forearm of a human volunteer. When engaged, no sensation was felt, but evidence of stratum corneum ablation was apparent when the skin was examined under an operating microscope. Subsequent treatments on the same position on the skin produced minimal sensation until a critical number of pulses had been applied, whereupon a slightly painful sensation was experienced. Enhanced skin ablation with increasing number of applied pulses was observed through the microscope.

This test was repeated with the needle, printed-circuit, conductive-ink and conductive tape electrodes. Visible ablation of the skin was apparent in all cases. The sensation experienced by the volunteer was slightly different depending on which electrode was used. The needle electrodes produced the least sensation while the printed-circuit and conductive-tape electrodes produced the greatest sensation. When an electrically insulating material such as Scotch tape was placed between the conductive-tape electrodes and the skin, the sensation was reduced.

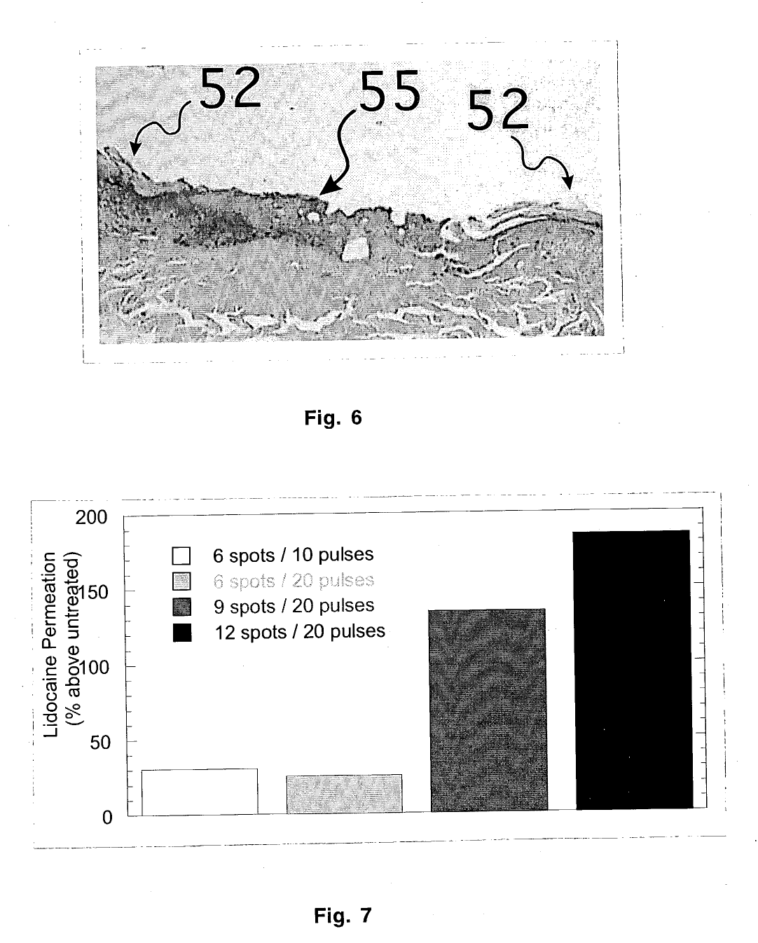

Human skin harvested from cadavers was purchased from a skin-bank and thawed to room-temperature before use. A conductive-ink electrode 40 as shown in FIGS. 5A or 5B, was positioned with gentle pressure against the stratum-corneum side of the skin. The treatment device was set to produce single pulses at the highest energy. Ten treatments were done on the same spot, before the tissue was removed and subsequently placed in 10% formalin. The sample was prepared using standard histological techniques, stained with hematoxylin-eosin stain and mounted on a microscope slide. A photomicrograph showing clear evidence of stratum corneum ablation 55 can be see in FIG. 6. The intact stratum corneum 52 can also be seen. There is no evidence of damage to the underlying dermis.

### EXAMPLE 3

Enhanced Permeation of Substances Through the Skin

A series of in vitro drug permeation tests were performed. The treatment device was connected to the needle electrodes, which were spaced approximately 2 mm apart. Human skin harvested from cadavers was purchased from a skin-bank and thawed before use. Samples of the split-thickness skin, approximately 15×15 mm, were positioned stratum-corneum side up, on the receptor chamber of water-filled Franz diffusion cells. The donor diffusion chamber was then positioned on top of the skin and was then sealed with an occlusive plastic film to prevent dessication of the skin. The entire test system, receptor and donor chamber, was positioned in a heated-stirring block which was maintained at 34° C. and which gently stirred the receptor chamber water. After being left overnight, the water in the receptor chamber was replaced with fresh water. The donor chamber was gently removed and the skin was patted dry prior to treatment with the device.

One spot on the skin was treated with a burst of 16 pulses, each separated by about 150 μs, and at a maximum pulse energy. This was repeated 10-20 times on each of 6, 9 or 12 separate spots on the skin. After treatment, the donor chamber was replaced on the skin and the whole diffusion cell assembly was replaced in the heated-stirring block. Replicates of skin were treated identically, and untreated controls were also produced.

At this time, a 50 μl test sample consisting of 4% lidocaine-HCl was applied in the donor chamber of all the cell assembly. The water in the receptor chamber was collected at 24 hours post-lidocaine application. The amount of lidocaine in the sample was measured using high-performance liquid chromatography (HPLC) followed by absorption spectrophotometry. The HPLC detection system was calibrated using samples with known lidocaine concentration. The results of this study, shown in FIG. 7, illustrate the enhanced permeation when the treatment device is used to increase the permability of the skin. Increased treatment,e.g. 12 spots of 20 repetitions versus 6 spots or 10 repetitions, clearly increases the permability of the skin.

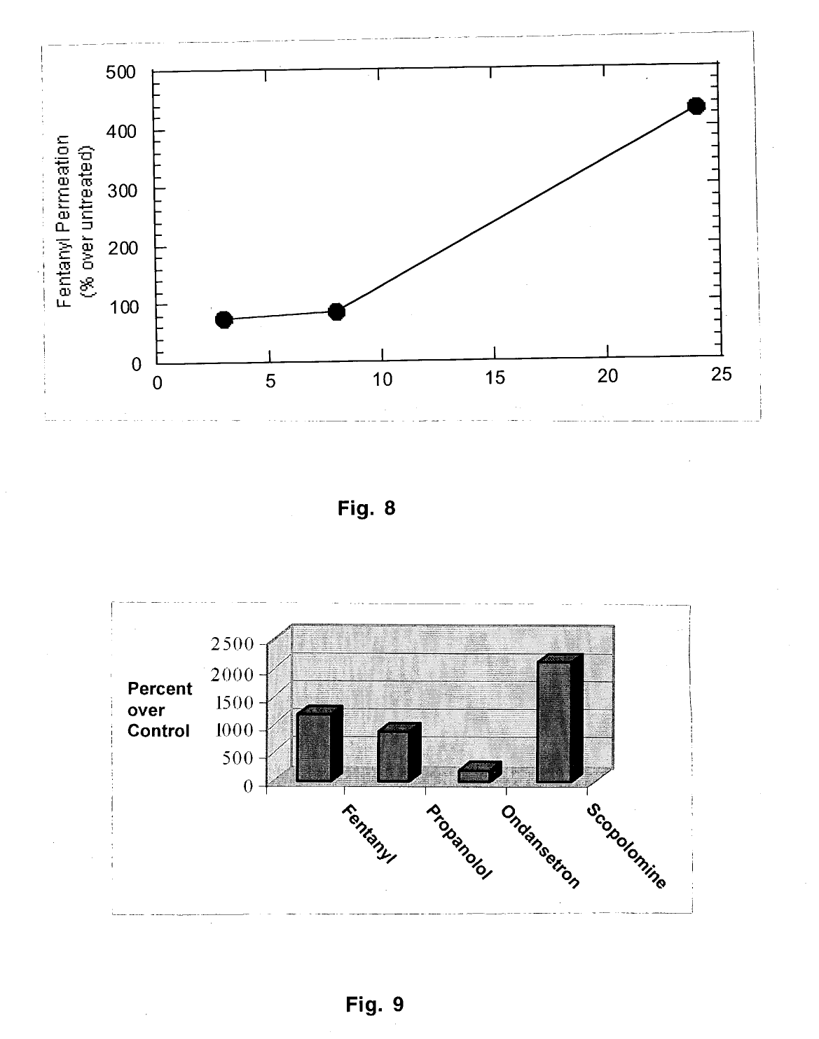

FIG. 8 shows the results of a similar permeation experiment done with 1.0 mg/ml fentanyl citrate after the skin was treated in six separate spots with 20 bursts of 16 pulses, each separated by about 150 μs, and at a maximum pulse energy. In this case, samples were taken from the receptor chamber at 3, 8 and 24 hours post fentanyl application. The results illustrate that the permeation increases with time, perhaps due to the finite time it takes fentanyl to penetrate the dermis prior to distributing in the water in the receptor chamber.

Additionally, enhanced permeation was demonstrated in vitro with fentanyl, propanolol, ondansetron and scopolamine, and treatment conditions of six separate spots with 20 bursts of 16 pulses, each separated by about 150 μs, and at a maximum pulse energy. The results of the total drug permeation enhancement after 24 hours post-drug-application is shown in FIG. 9.

The following reference is cited herein:

1. Rand et al. J. Arthro. Surg. Vol. 1, pgs. 242-246 (1985).

Any patents or publications mentioned in this specification are indicative of the levels of those skilled in the art to which the invention pertains. These patents and publications are herein incorporated by reference to the same extent as if each individual publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A device for increasing the rate of permeation of a biological substance through a biomembrane of an individual comprising:
an active electrode, said active electrode comprising a proximal end and a distal end, wherein said active electrode delivers a high frequency voltage to the biomembrane; and
a return electrode, said return electrode located distally to said active electrode.

2. The device of claim 1, further comprising a control means, said control means independently controlling current flow from said active electrode wherein impedance between said active electrode at a target site on the biomembrane and said return electrode determines said current flow.

3. The device of claim 2, wherein said control means generates a high electric field intensity at the distal end of said active electrode.

4. The device of claim 3, wherein said electric field intensity causes molecular disruption of necrotic or dead tissue, biomolecules or cells at a target site on the biomembrane of said individual.

5. The device of claim 1, wherein said active electrode is moved over said target site on the biomembrane of the individual during delivery of the voltage.

6. The device of claim 5, wherein said target site comprises an area of about 0.1 cm² to about 20 cm².

7. The device of claim 1, wherein said active electrode and said return electrode comprise a coaxial electrode, needles, a printed-circuit, conductive-ink or conductive tape.

8. The device of claim 7, wherein said conductive ink or said conductive tape is positioned on an electrically insulative material, said material placed on the biomembrane, wherein said insulative material insulates that part of the biomembrane from said conductive ink or said conductive tape not in immediate contact with a target site on the biomembrane.

9. The device of claim 1, wherein the active electrode comprises an electrode array, said array comprising a plurality of isolated electrode terminals.

10. The device of claim 1, wherein said active electrode and said return electrode are in a patch.

11. The device of claim 10, wherein the active electrode comprises a transducer.

12. The device in claim 1, further comprising a safety interlock, said interlock allowing or preventing operation of said device.

13. The device of claim 1 further comprising a container, said container located at the distal end of said active electrode wherein said container optionally is integral with said safety interlock.

14. The device in claim 1, further comprising a means to apply an electrically conductive or electrically insulating fluid.

15. The device of claim 14, wherein the electrically conductive or electrically insulating fluid contains said biological substance.

16. The device of claim 1, wherein said substance is a pharmaceutical compound.

17. The device of claim 16, wherein said pharmaceutical compound is nitroglycerin, an anti-nauseant, an antibiotic, a hormone, a steroidal antiinflammatory agent, a non-steroid antiinflammatory agent, a chemotherapeutic agent, an anti-cancer agent, an immunogen, an analgesic, an anti-viral agent or an anti-fungal agent.

18. The device of claim 17, wherein said anti-nauseant is scopolamine.

19. The device of claim 17, wherein said antibiotic is tetracycline, streptomycin, sulfa drugs, kanamycin, neomycin, penicillin, or chloramphenicol.

20. The device of claim 17, wherein said hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin.

21. The device of claim 1, wherein said biological substance is interstitial fluid.

22. The device of claim 21, wherein said interstitial fluid is collected for the purpose of measuring analytes.

23. The device of claim 1, wherein said substance is a diagnostic material.

24. A method of increasing the rate of permeation of a substance through the skin of an individual comprising the steps of:
applying a high frequency voltage with the device of claim 1 to a target area on the skin of said individual, wherein said target area on the skin comprises a substance applied externally on or located internally to said target area; and
reducing the stratum corneum of the skin with the successive application of said high frequency voltage thereby increasing the rate of permeation of the substance through the skin of said individual.

25. The method of claim 24, further comprising the step of moving said active electrode over said target site on the skin of said individual during the application of the voltage.

26. The method of claim 24, wherein said internal substance is withdrawn from the target area on the skin of said individual.

27. The method of claim 26, wherein said substance is interstitial fluid.

28. The method of claim 27, wherein said interstitial fluid is collected for the purpose of measuring analytes.

29. The method of claim 24, wherein said substance is a diagnostic material.
